(19) United States
(12) Patent Application Publication
(10) Pub. No.: US 2024/0315591 A1
(43) Pub. Date: Sep. 26, 2024

(54) DETECTION OF RESPIRATORY TRACT INFECTIONS (RTIS)

(71) Applicant: NANOSE MEDICAL LTD, Isfiya (IL)

(72) Inventors: Orit MAROM ALBECK, Herzliya (IL); Osnat SELLA TAVOR, Kfar Kisch (IL)

(21) Appl. No.: 18/259,829

(22) PCT Filed: Jan. 6, 2022
(86) PCT No.: PCT/IL2022/050021

§ 371 Date: Jun. 29, 2023

Related U.S. Application Data
(60) Provisional application No. 63/134,627, filed on Jan. 7, 2021.

(51) Int. Cl. A61B 5/08; A61B 5/00; A61B 5/097; G01N 33/00; G01N 33/497

(52) U.S. Cl. CPC A61B 5/082; A61B 5/097; A61B 5/7264; G01N 33/0047; G01N 33/497

(57) ABSTRACT

The invention generally concerns method of diagnosis of respiratory tract infections by identifying presence of volatile compounds (VCs) and other relevant markers in vapor and other bodily samples collected form subjects suspected of having the infection.

### TECHNOLOGICAL FIELD

The present invention is generally directed to the diagnosis of respiratory tract infections by identifying presence of volatile compounds (VCs) and other relevant markers in vapor and other bodily samples collected form subjects suspected of having a respiratory infection.

### BACKGROUND ART

Volatile compounds (VCs), in general, and specifically volatile organic compounds (VOCs), constitute endogenous products of metabolic processes that are produced by the body during an inflammatory response that accompanies bacterial infections and also released from bacteria cells during infection of the respiratory system. VCs are transported from different organs via blood to the lungs and subsequently excreted from the lungs by diffusing across the pulmonary alveolar membrane and exhaled via the breath.

VCs represent a wide range of stable chemicals that are volatile at ambient temperature and are detectable in exhaled breath, urine, feces, blood and sweat and assessment of endogenous VCs can provide insights into healthy and diseased metabolic states, whereas the detection of exogenous compounds suggests exposure to a drug or compound associated with environmental or occupational exposure.

Noninvasive methods for the detection of VOCs, like breath analysis, are preferred for diagnosis due to the ease of sampling procedures and low cost, the breath can be sampled and analyzed in real time [1].

Ashrafi M et al [2] reported on the validation of biofilm formation on human skin wound models and demonstration of clinically translatable bacteria-specific volatile compound signatures comprising ethanol, 2-butanol, 2-methyl-1-propanol, 3-methyl-1-butanol and 1-undecane.

Lai S Y et al [3] reported on the identification of upper respiratory bacterial pathogens with the electronic nose.

Bos L D et al [4] reported on volatile compounds/metabolites of pathogens, including Strep pneumonia and some of the indicated volatile molecules in this document are indicated there along with their relevant metabolic pathways.

US patent public cation no. 2012/326,092 [5] provides methods of diagnosis, prognosis and monitoring of various types of cancer by determining the levels of signature sets of volatile organic compounds (VOCs) in a breath sample, wherein significantly different levels of said VOCs compared to a control sample are indicative for the presence of either one of breast, head and neck, prostate and colon cancers.

### PUBLICATIONS

[1] Zhang R, et al., 2017 Anal Chem 89: 3353-61,
[2] Ashrafi M, et al., Sci Rep. 2018; 8(1):9431,
[3] Lai S Y, et al., Laryngoscope 112, 975-9 (2002),
[4] Harbeck M, et al., PLOS Pathog. 2013; 9(5),
[5] US Patent Publication No. 2012/326,092.

### SUMMARY OF THE INVENTION

The diagnosis of respiratory tract infections typically requires obtaining samples from lower regions of the respiratory tract that are difficult to reach or, for throat infections, sample collection with a throat swab and laboratory culture systems, which, while are highly sensitive, require support of a full microbiology laboratory and relatively long processing time to produce results. In addition, a high cost may be involved with laboratory base systems and the systems are typically not portable.

The inventors of the present invention have developed a diagnostic method permitting diagnosis or prediction of evolution and occurrence of bacterial infection in the respiratory tract by detecting presence of volatile compounds (VCs, such as volatile organic compounds, VOCs) in exhaled breath or in biological samples obtained from the subject. Samples collected from subjects suspected of having a respiratory tract bacterial infection (RTIs) have been found to contain such VCs which presence and amount was indicative of an early stage infection, and could therefore be used to provide an indication of progression of the infection and also of the efficacy of treatment. As methods of the invention permit distinguishing bacterial infections from viral infection, the same methods may also be suitable for detecting VCs associated with viral infections.

The herein described method is non-invasive, does not require throat swab and complex analysis, is of low cost and can provide an efficient means to diagnose and perform treatment monitoring by physicians or any other staff like nurses, pharmacists or lab technician at any point of care, such as at the physician clinic or office, in emergency care units, clinical labs or pharmacies as well as in a patient's home.

The method of the present invention relies on the finding that exhaled breath as well as biological samples collected from subjects include volatile materials that are present in different levels and compositions in samples obtained from patients having a bacterial infection of the upper or a lower respiratory tract as compared to individuals not suffering from such an infection. The presence of a unique set of VCs, being in levels and compositions that are different from VCs in individuals not having the bacterial infection, or who are suffering from viral infections, enables to selectively and with high precision differentiate between bacterial infections and viral infections of the upper or lower respiratory tract.

Thus, according to a first aspect, the present invention provides a method of determining presence of a bacterial infection of the respiratory tract (of the upper or lower regions of the tract) in a subject, the method comprising:
a) determining a volatile compound (VC) profile in a breath sample or headspace of a biological sample collected from a subject suspected of having the infection; and
b) comparing the VC profile to a VC profile of a control, as defined herein, and/or optionally to a VC profile of a sample obtained from the subject at an earlier time point(s), as defined herein;
wherein a VC profile different from the control VC profile is indicative of one or more of (1) presence of the bacterial infection, (2) absence of the bacterial infection, or (3) a change (e.g., an improvement or worsening) of the bacterial infection.

The invention further provides a method for differentiating between a bacterial infection and a viral infection of the respiratory tract in a patient, the method comprising:
a) determining a volatile organic compound (VC) profile in a breath sample or a biological sample collected from the subject suspected of having the infection; and
b) comparing the VC profile to a VC profile of a control and/or optionally to a VC profile of a sample obtained from the subject at an earlier time point(s).

Further provided is a method of determining presence of a bacterial infection of the respiratory tract (of the upper or lower regions of the tract) in a subject, the method comprising:
a) exposing a gaseous sample (a breath sample or a headspace sample, as defined herein) comprising volatile compounds (VCs) to a sensor responsive to interaction with the volatile compounds;
b) detecting/measuring an output signal received from the sensor correlating with an interaction between the VCs and the sensor; and
c) using a learning and pattern recognition algorithm to determine presence of a pattern of volatile compounds indicative of bacterial infection.

Further provided is a method of determining a respiratory tract bacterial infection in a subject, the method comprising:
a) exposing a gaseous sample being a breath sample or a headspace sample comprising volatile compounds (VCs) to a sensor responsive to interaction with the volatile compounds;
b) detecting/measuring an output signal received from the sensor correlating with an interaction between the VCs and the sensor; and
c) determining presence of a pattern of volatile compounds indicative of bacterial infection.

In accordance with methods of the invention, where the sample comprises VCs that are unique in pattern or profile (considering type of VCs, composition of VCs, amount of VCs, etc, as further defined herein) to bacterial pathogens present in the respiratory system, such a sample will provide a determination or an indication of presence of the bacterial infection. The unique pattern of VC indicative of bacterial infection may be determined using a learning and pattern recognition algorithm. This algorithm enables learning, dimensionality reduction, classification, regression, optimization and pattern recognition.

In some embodiments, the algorithm may be selected from artificial neural network algorithms (ANN), gradient descent, spike timing dependent plasticity (STDP), principal component analysis (PCA), multi-layer perception (MLP), generalized regression neural network (GRNN), convolutional neural networks (CNN), spiking neural networks (SNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, k nearest neighbors (KNN), k means clustering (K means), spectral clustering, support vector machine (SVM), logistic regression, and random forest and naïve Bayes.

In some embodiments, the algorithm is LDA.

In some embodiments, the method comprises obtaining a device comprising
a sample collecting chamber;
at least one sensor assembly comprising one or a plurality of sensing regions, wherein the assembly is in gaseous communication with said sample collection chamber;
a closed loop channel assembly for directing said sample from the sample collecting chamber to the at least one sensor assembly and for circulating said sample from the sample collecting chamber over the at least one sensor assembly over a period of time;
optionally means for circulating the sample, e.g., a pump; and
a pattern recognition analyzer (e.g., configured for real-time analysis of a VC profile derived from content of the sample);
wherein the method is carried out on the device.

As used herein, a “sensor responsive to interaction with the volatile compounds” is any sensor capable of generating a signal upon coming in contact with a volatile compound. The interaction and thus generation of a signal indicative of said interaction may be immediate or may be concentration dependent. Typically, the sensor used in devices of the invention is such which permits immediate signal generation indicative of the interaction. The sensor may be provided in a form of at least one sensor assembly that is selected to be responsive and interact with VCs characteristic of bacterial infections of the respiratory tract.

In some embodiments, the sensor is a sensor assembly.

In some embodiments, the at least one sensor assembly comprises one or more chemically sensitive sensors and a processing unit comprising a learning and pattern recognition analyzer configured for receiving sensor output signals and comparing the signals to a stored data. In some embodiments, the analyzer is selected to enable learning, dimensionality reduction, classification, regression, optimization and pattern recognition.

In some embodiments, the sensor or sensor assembly is selected to be responsive and interact with VCs characteristic of bacterial infections of the respiratory tract.

In some embodiments, the sensor or sensor assembly is selected to be responsive and interact with VCs characteristic of bacterial infections of the respiratory tract and substantially unresponsive or less responsive or differently responsive to VCs characteristic of viral infections.

In some embodiments, the sensor or sensor assembly is selected to be responsive and interact with VCs selected from ethanol, methanol, 2-butanol, pentanol, 2-methyl-1-propanol, 3-methyl-1-butanol, 2-methyl-butanal, 1-undecane, 2,4-dimethyl-1-heptane, 2-butanone, 2-propanol, 4-methyl-quinazoline, 1-octanol, ethyl acetate, lactic acid, isovaleric acid, indole, hydrogen cyanide, methyl thiocyanide, 2-acetophenone, ammonia, methylthiocyanide, 2,2,4,4-tetramethyloxolane, methyl-4-methylpentanoate, 4-methyl pentanoic 4-acid, 1-methyl-2-(1-methylethyl)-benzene, cymol, methyldodecane, methyl nicotinate, 1,2-bis(trimethylsily)-benzene, gamma-butyrolactone, 3Z-octenyl acetate, 3-methylcyclo hexene, superoxide anion (O₂·—), hydroxyl radicals (OH·), singlet oxygen (O₂·), hydrogen peroxide (H₂O₂), hypochlorous acid (HOCI) and myeloperoxidase (MPO).

In some embodiments, a sensor or a sensor assembly suitable for interaction with VCs according to the invention may be in a form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

In some embodiments, the sensor is or comprises nanoparticles.

In some embodiments, the sensor is provided in the form of a plurality of nanoparticles associated to a surface. The sensor surface may comprise one or more sensing regions, each of the regions being associated with same or different population of nanoparticles, such that a signal may be independently derived from each of the sensing areas, and be indicative of an interaction (or lack thereof) between VCs present in the sample and the nanoparticles on the sensing regions.

Each of the sensing regions present on the sensor surface comprises a plurality of nanoparticles of a particular population, wherein each population differs from another in at least one of particle size, particle morphology (e.g., core/shell particles, non-core/shell particles, spherical, cubic, tetrahedral, triangular, dumbbell, elongated, multiparticles or fused particles, etc), particle composition (e.g., doping, metallic particles, non-metallic particles, conductive particles, novel metal particles, hybrid materials, etc), surface decoration (e.g., presence of material islands, association with ligand groups, etc) and others.

In some embodiments, the sensor surface comprises one or more sensing regions, each of the regions being associated with same or different population of nanoparticles, wherein a signal independently derived from each of the sensing areas is indicative of an interaction (or lack thereof) between VCs present in the sample and the nanoparticles on the sensing regions.

In some embodiments, each of the sensing regions comprises a plurality of nanoparticles of a particular population, wherein each population differs from another in at least one of particle size, particle morphology, particle composition, and surface decoration.

In some embodiments, each sensing region comprises a different selection of nanoparticles. In some embodiments, each sensing region comprises a mixed population (an inhomogeneous population) of nanoparticles, while in other embodiments, each sensing region comprises a uniform population (a homogenous population) of nanoparticles.

In a plurality of such sensing regions, one or more thereof may comprise a plurality of particle populations, namely an inhomogeneous population of particles, wherein some of the nanoparticles differ in structure, others in composition and still others in surface decoration. For example, a sensing region may comprise two populations of nanoparticles, one population comprising particles of one metal and another population comprises particles of a different metal. In a similar way, all particles may be of one metal but differ from each other in their surface decoration (e.g., presence of ligands or selection of ligands).

In some embodiments, the nanoparticles are core/shell particles, non-core/shell particles, spherical, cubic, tetrahedral, triangular, dumbbell, elongated or fused particles. In some embodiments, the particles are spherical in shape.

In some embodiments, the nanoparticles are metallic nanoparticles. In some embodiments, the metallic nanoparticles are of at least one metal optionally selected amongst any metal of the Periodic Table of the Elements. In some embodiments, the metals are of any of Groups IIIB, IVB. VB, VIB, VIIB, VIIIB, IB and IIB of block d of the Periodic Table. In some embodiments, the metal is selected from Sc. Ti, V, Cr, Mn, Fe, Ni, Cu, Y, Zr, Nb, Tc, Ru, Mo, Rh, W, Au, Pt, Pd, Ag, Au, Al, Mn, Co, Cd, Hf, Ta, Re, Os. Ir and Hg.

In some embodiments, the metal is gold, silver, nickel, cobalt, copper, palladium, platinum or aluminum. In some embodiments, the nanoparticles are gold nanoparticles.

The metallic nanoparticles may or may not be doped or further comprise an amount of another metallic or non-metallic material. The metallic nanoparticles may be bare, namely uncoated, or coated with a plurality of surface associated ligand molecules. Such ligand molecules may have surface anchoring groups which may vary based on, e.g., the composition of the nanoparticles. For example, where the nanoparticles are gold nanoparticles, the surface anchoring groups may be a thiol, a disulfide, an amine and others as known in the art.

In some embodiments, wherein the nanoparticles are gold nanoparticles.

In some embodiments, methods of the invention are carried out on a breath sample obtained from the subject, wherein the breath sample is obtained as detailed herein. In some embodiments, the breath sample is a non-alveolar breath sample.

In some embodiments, the sample is a biological sample (e.g., a blood sample, a urine sample, feces, a sweat sample or a sample of saliva), wherein VCs are measured in a headspace of said sample.

In some embodiments, the biological sample is maintained in a closed container and headspace gases are collected and subsequently measured.

As noted above, the method is carried out on a subject suspected of having a bacterial infection of the lower respiratory tract infection or the upper respiratory tract. The method may be similarly applied to subjects (humans and non-humans) who are predisposed to attracting the infection, subjects who routinely suffer from the infection or subjects who have contracted the infection, have shown symptoms associated with the infection and have been treated.

In some embodiments, the method comprises obtaining a breath sample from a subject by employing any non-invasive means known in the art. In order to minimize inclusion of VCs that are not related to diseases or conditions of the respiratory tract, the breath sample may be obtained, e.g., by suction, from the lower regions of the tract or from the oral cavity of the subject, or by relaxed exhalation of air. In some embodiments, where the diagnosis relating to the upper respiratory tract is desired, the breath sample may be a non-alveolar breath sample.

Non-limiting methods for collecting such exhaled breath may involve the use of apparatuses approved by the American Thoracic Society/European Respiratory Society (ATS/ERS), see for example Silkoff et al., Am. J. Respir. Crit. Care Med., 2005, 171, 912, or may involve direct exhalation of breath into a measuring device or apparatus.

Where the sample is a biological sample, such as a blood sample, a urine sample, feces, a sweat sample or a sample of saliva, VCs are allowed to evaporate into a headspace above the liquid or semisolid sample and are collected and analyzed. Evaporation may be overtime or may be hastened by slightly heating the sample to cause evolution of the volatile components in the sample.

In some embodiments, the sample, being a breath sample or a headspace sample, is captured into a container (e.g., a syringe, a vessel) which may be stored until analysis using the herein described method. Where the sample is a breath sample, it may be collected by directly exhaling breath onto the device and/or sensor of the invention. According to such embodiments, the breath sample may be captured using a mouthpiece that provides an interphase between the subject and the device operated according to methods of the invention to enable analysis of the breath sample by the sensor of the invention.

As the concentration of VCs in the sample, e.g., human breath, may be in the range of ppm to ppt, the method may further comprise a step of pre-concentrating the obtained sample prior to analysis. Sample concentrators that are within the scope of the present invention include but are not limited to those described in US 2012/0326092 which is herein incorporated by reference.

Bacterial infections of the respiratory tract generally refer to illnesses or medical conditions caused by an acute infection, involving the upper respiratory tract, including the nose, sinuses, pharynx and the larynx, or the lower respiratory tract, including the trachea and the lungs (including the bronchi, bronchioles and the alveoli). Bacterial infections of the upper respiratory tract, known as upper respiratory tract infections (URTIs) include bacterial pharyngitis, viral pharyngitis, tonsillitis, laryngitis, epiglottitis, tracheobronchitis, sinusitis, otitis media and the common cold. Bacterial infections of the lower respiratory tract, known as lower respiratory tract infections (LRTIs) include bacterial bronchitis, pneumonia and bronchiolitis. These and other URTIs/LRTIs (generally referred to as RTIs) may be caused, induced or brought about by one or more bacterial pathogen such as Staphylococcus aureus, Streptococcal Pyogenes, Pseudomonas aeruginosa or Escherichia coli.

Within the context of the present invention, volatile compounds (VOC) are compounds, typically but not necessarily volatile organic compounds (VOCs), that are associated with the metabolism, presence and/or growth of at least one pathogen (e.g., bacteria or virus) involved in the pathogenesis of an upper respiratory infection. VCs that are generated in the body, e.g., through the metabolism of cells or pathogens within the body, are released into the circulatory system and thereafter excreted through the skin, the urine, saliva blood and/or exhaled breath. The VCs may comprise a plurality of compounds, some of which gaseous, others may be liquids (at a physiological temperature), which are released into the excreted biological sample or breath, and thus can be detected and quantified. When released via exhaled breath, the VCs may be carried by the breath gases or small droplets of water.

The “VC profile” refers to a measured signature or an electronic signal pattern or electronic signature derived from a collection of properties relating to the VC content of the sample, e.g., exhaled breath, which is indicative of the presence/absence of a bacterial infection and which enables differentiation between infections or diseases that are caused by a bacterium and such which are viral or others. These collective properties are unique to samples obtained from infected subjects and are thus informative, transformed into an electric signal or a fingerprint or a signature that can provide an indication of onset, evolution or progression of a bacterial respiratory tract infection. The VC profile can also provide an insight as to the state of the infection or the progression thereof, can identify the onset of the disease at an early stage before symptoms develop and can assist in determining success of a therapeutic treatment (prophylaxis or treatment of existing symptoms). The signal patterns derived from the collective properties may vary based on one or more of:
presence or absence of one or more VCs indicative of the infection,
the concentration (or amount) of the one or more VCs,
the presence or absence of other VCs in combination,
the ratio amounts between the various VCs, and
a change in the presence or amount of one or more VCs over time.

In methods and systems of the invention, and as disclosed hereinbelow, a pattern recognition module or analyzer is used to generate signal patterns that are characteristics of the VC profile (materials, amounts, ratios, etc). For the purpose of defining a VC profile or a signature indicative of VCs associated with presence of a bacterial infection in the respiratory tract, knowing the nature and amount of the VCs is not necessary.

In most general terms, however, sensors utilized according to the invention are configured to interact and respond to the presence of VCs indicative of the onset, presence or evolution of a bacterial infection, i.e., URTI or LRTI, such as ethanol, methanol, 2-butanol, pentanol, 2-methyl-1-propanol, 3-methyl-1-butanol, 2-methyl-butanal, 1-undecane, 2,4-dimethyl-1-heptane, 2-butanone, 2-propanol, 4-methyl-quinazoline, 1-octanol, ethyl acetate, lactic acid, isovaleric acid, indole, hydrogen cyanide, methyl thiocyanide, 2-acetophenone, ammonia, methylthiocyanide, 2,2,4,4-tetramethyloxolane, methyl-4-methylpentanoate, 4-methyl pentanoic acid, 1-methyl-2-(1-methylethyl)-benzene, cymol, 4-methyldodecane, methyl nicotinate, 1,2-bis(trimethylsily)-benzene, gamma-butyrolactone, 3Z-octenyl acetate, 3-methylcyclo hexene, superoxide anion (O₂·—), hydroxyl radicals (OH·), singlet oxygen (O₂·), hydrogen peroxide (H₂O₂), hypochlorous acid (HOCI) and myeloperoxidase (MPO).

In some embodiments, methods of the invention aim at determining onset, presence or evolution of at least one bacterial infection of the upper respiratory tract (URTI). In some embodiments, methods of the invention aim at determining onset, presence or evolution of at least one bacterial infection of the lower respiratory tract (LRTI). In some embodiments, methods of the invention aim at determining onset, presence or evolution of at least one bacterial infection induced by Streptococcal pyogenes. In some embodiments, the URTI is pharyngitis.

In some embodiments, the method of the invention aims at determining onset, presence or evolution of pharyngitis. In one embodiment, the pharyngitis is bacterial pharyngitis. In some embodiments, bacterial pharyngitis may be caused by Streptococcus pyogenes, Streptococcus pneumoniae, Haemophilus influenzae, Corynebacterium diphtheriac, Bordetella pertussis or Bacillus anthracis, and thus the presence of each of these pathogens in the upper respiratory tract may be detected using methods of the invention. In some embodiments, the bacterial pharyngitis is caused by Streptococcus pyogenes or Streptococcus pneumoniae. In some embodiments, the pathogen is Streptococcus pyogenes.

In some embodiments, the VC profile indicative of bacterial pharyngitis is based on the presence of at least one VC selected from isovaleric acid, 2-methyl-butanal, 1-undecene, 2,4-dimethyl-1-heptane, 2-butanone, 4-methyl-quinazoline, hydrogen cyanide, methyl thiocyanide, methanol, pentanol, ethyl acetate and indole.

In some embodiments, the VC profile indicative of bacterial pharyngitis is based on the presence of at least one VC selected from ammonia, superoxide anion, hydroxyl radicals, singlet oxygen, hydrogen peroxide, hypochlorous acid and myeloperoxidase (MPO).

In some embodiments, the VC profile indicative of the presence of Streptococcal pyogenes is based on one or more VCs that are associated with the metabolism, presence and/or growth of Streptococcal pyogenes. Such VCs may be associated with a biochemical pathway such as phosphoenol pyruvate (PEP)-dependent phosphotransferase (PTS) pathway, catabolite repression and Embden-Meyerhof-Parnas (EMP) pathway.

In some embodiments, the VCs are those uniquely indicative of the onset, presence and evolution of bacterial pharyngitis, but not of rheumatic fever, rheumatic heart disease or scarlet fever. In some embodiments, VC profile indicative of bacterial pharyngitis is based on the presence of at least one VC selected from 2-methylbutanol, 3-methylbutanol, 2-butanol, lactic acid, acetic acid and indole.

In some embodiments, a VC profile indicative of the onset, presence and evolution of an infection associated or induced by Streptococcal pyogenes is based on the presence of 1, 2, 3, 4, 5, 6, 7 or 8 VCs. In some embodiments, the ratio between any two VCs in a combination of two or more VCs may be between 0.0001:1 and 1:0.0001. For example, in cases where the VC profile comprises two VCs, e.g., VC-A and VC-B, the ratio between the two may be between 0.0001:1 and 1:0.0001. In cases where three VOCs are present, e.g., VC-A, VC-B and VC-C, the ratio between VC-A and VC-B, the ratio between VC-A and VC-C and the ratio between VC-B and VC-C may be each between 0.0001:1 and 1:0.0001.

The electronic signal or pattern defining a VC profile indicative of a bacterial infection is compared with an electronic signal or pattern defining a “control” sample or a plurality of samples, which may be characteristic of (i) a healthy subject population, namely a population that is not diseased, (ii) a population of subjects who have been tested and found not to have been infected with a bacterial infection, or known to be free of a bacterial infection—this population being regraded herein as the “negative group”, and/or (iii) a population of subjects who are suffering from a bacterial infection in the respiratory system-herein rerefer to as the “positive group”. When comparing a signal indicative of the VC profile to a signal indicative to a VC profile of a positive and negative groups, a determination can be made whether the subject has or does not have the bacterial infection.

Control samples obtained for the purpose of determining the presence or absence of a bacterial infection are typically taken from a plurality (one or more) of subjects which have been identified as healthy (not having the disease-thus a negative group) or as sick (who have contracted the diseases-thus a positive group). The number of subjects may be at least 10, 20, 30, 40, 50, 60, 70, 80, 90, 100, 150, 200, 250 to thousands of subjects.

Where determining progression of the disease, one or more VC profiles may be obtained for a group of subjects suffering from a disease, wherein each profile is obtained at a different time point along the way to recovery.

Where the subject is a human, the control is a human, and where the method if used on non-human mammals, the control group should include species from the same group.

In some embodiments, the VC profile is determined by an E-nose or according to the method described in US 2012/0326092, herein incorporated by reference.

A change in an electronic signal generated by a pattern recognition module based on a particular VC composition, may be determined by utilizing an algorithm such as, but not limited to, neural network algorithms (ANN), gradient descent, spike timing dependent plasticity (STDP), principal component analysis (PCA), multi-layer perception (MLP), generalized regression neural network (GRNN), convolutional neural networks (CNN), spiking neural networks (SNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, k nearest neighbors (KNN), k means clustering (K means), spectral clustering, support vector machine (SVM), logistic regression, and random forest and naïve Bayes. Using such algorithms or others known in the art, a signal generated for the VC profile defining a sample obtained from a subject may be regarded as being significantly different from a signal generated for a VC profile of a control group, thus providing an indication of presence or absence of a bacterial infection. A statistically significant difference can be determined by any test known to the person skilled in the art. Common tests for statistical significance include, among others, t-test, ANOVA1 Kruskal-Wallis, Wilcoxon, Mann-Whitney and odds ratio.

Statistical significance may alternatively be calculated as P<0.05. Methods of determining statistical significance are known and are readily used by a person of skill in the art. In a further alternative, increased levels, decreased levels, deviation, and changes can be determined by recourse to assay reference limits or reference intervals Overall, these methods calculate the 0.025, and 0.975 fractiles as 0.025*(n+1) and 0.975*(n+1). Such methods are well known in the art.

Various other algorithms are known in the art, which are disclosed, for example, in U.S. Pat. Nos. 6,411,905, 6,606,566, 6,609,068, 6,620,109, 6,767,732, 6,820,012 and 6,839,636, each of which being incorporated herein by reference.

In accordance with the present invention, the VC profile indicative of a bacterial infection comprises VCs that are present in breath samples or other biological samples from patients suffering from the infection, in levels which are at least one standard deviation [SD] larger or smaller than their mean level in breath samples of a negative control population. In some embodiments, the levels of VCs in breath samples of infected patients are at least 2 standard deviations[SD] or 3[SD] larger or smaller than their mean level in breath/saliva/nasal secretion samples of a negative control population. Accordingly, samples (of unknown status) are considered to belong to a positive population (i.e., suffering from the infection) when the level of VCs is at least 1[SD]. 2[SD] or 3[SD] larger or smaller than the mean level of VCs in breath samples of a negative control population.

In some embodiments, the level of the one or more VC in the sample is significantly increased as compared to the level of the VC in a control. In some other embodiments, the level of the one or more VC in the sample is significantly decreased as compared to the level of the VC in a control. In some embodiments, the levels of the one or more VC in the sample collected from the patient having the infection form a pattern which is significantly different from the pattern of the VCs in the control. In some embodiments, the levels of the one or more VC in the sample collected from the patient having URTI form a pattern which is significantly different from a predetermined pattern of occurrence of VCs in breath samples taken from subjects who are not suffering from the disease.

In accordance with the present invention, the difference in the VC profile between a sample collected from a patient suspected of having an infection and a control or a sample collected from a subject not having the infection can be analyzed using various pattern recognition modules or analyzers commonly used in the art and as described, for example, in US 2012/0326092 which is herein incorporated by reference.

Also provided herein is a VC from a sample, e.g., a breath sample, for use in diagnosis, prognosis and/or monitoring of an infection of the respiratory tract (e.g., bacterial pharyngitis), monitoring disease progression and treatment efficacy. The diagnosis, prognosis and/or monitoring of the infection comprises the diagnosis of a subject who is at risk of developing the infection, a subject who is suspected of having the infection, or a subject who was diagnosed with infection using commonly available diagnostic tests.

The invention further provides a method of treating a bacterial infection of a subject respiratory tract, the method comprising determining presence of the invention employing any of the methods of the invention and treating the subject, e.g., with an antibiotic, in case a determination is made that the subject has contracted the infection.

In most general terms, the therapeutic treatment may or may not involve administration of an antibiotic. The treatment may involve any medication or treatment regimen determined suitable by a medical practitioner.

Thus, a method for treating a bacterial infection in a subject suspected of contracting a bacterial infection of the respiratory tract, the method comprises
a) exposing a gaseous sample obtained from the subject, the sample being a breath sample or a headspace sample comprising volatile compounds (VCs), to a sensor responsive to interaction with the volatile compounds;
b) detecting/measuring an output signal received from the sensor correlating with an interaction between the VCs and the sensor;
c) determining presence bacterial infection;
wherein if bacterial infection is present, treating said subject, e.g., with an antibiotic.

In some embodiments, the method comprises obtaining the sample from the subject, as detailed herein.

In some embodiments, the method utilizes a device as detailed herein.

In some embodiments, the bacterial infection is of the upper respiratory tract.

In some embodiments, the bacterial infection is of the nose, sinuses, pharynx and the larynx.

In some embodiments, the bacterial infection is of the lower respiratory tract.

In some embodiments, the bacterial infection is of the trachea and the lungs.

In some embodiments, the bacterial infection is pharyngitis, viral pharyngitis, tonsillitis, laryngitis, epiglottitis, tracheobronchitis, sinusitis, otitis media, bacterial bronchitis, pneumonia and bronchiolitis

The invention further provides a device for carrying out methods according to the invention. The device of the invention may be any device for measuring/detecting components of exhaled breath of a subject which comprises a collection chamber for collecting, holding or communicating a volume of an exhaled breath to a sensor that can produce a unique (e.g., electronic) fingerprint to enable the determination of a VC profile from breath samples as described herein. Some non-limiting examples of sensor that can be used in accordance with the present invention includes functionalized surface regions (wherein such surfaces are functionalized with metal nanoparticles, functional molecules, hollow fibers and others), sensors having a functionalized nanowire or a nanotube, a polymer-coated surface acoustic wave (SAW) sensors, sensor employing a semiconductor gas sensor technology, aptamer biosensors, amplifying fluorescent polymer (AFP) sensors and others.

In accordance with the present invention, the sensor may be commercially referred to as an “artificial nose” or as an “electronic nose” which can non-invasively measure at least one VC in the exhaled breath and/or monitor the concentration of at least one VC in the exhaled breath of a subject as described herein. Thus, the herein described sensors enable qualitative and/or quantitative analysis of volatile compounds (e.g., gases, vapors, or odors) hence facilitates the device to carry out a method of the invention.

In some embodiments, the device comprises one or more (an array) of chemically sensitive sensors and a processing unit comprising a learning and pattern recognition analyzer configured for receiving sensor output signals and comparing the signals to a stored data, by utilizing a pattern recognition algorithm.

In some embodiments, the device may be a device disclosed in International Publication No. WO 2009/144725, herein incorporated by reference.

In some embodiments, the device utilizes a sensor as disclosed in US 2011/0269632, herein incorporated by reference.

In another aspect, there is provided a device that is optionally a hand-held device for determining presence of a bacterial infection in a subject, as defined herein, or for distinguishing bacterial infection over viral infection (or another disease), the device comprising
at least one sample collecting chamber;
at least one sensor assembly comprising one or a plurality of sensing regions responsive to interaction with volatile compounds present in a sample obtained from the subject, wherein the assembly is in gaseous communication with said sample collection chamber;
a closed loop channel assembly configured for directing said sample from the sample collecting chamber to the at least one sensor assembly and for circulating said sample from the sample collecting chamber over the at least one sensor assembly over a period of time;
and a pattern recognition analyzer (e.g., configured for real-time analysis of a VC profile derived from VC content of the sample).

In some embodiments, the at least one sensor assembly comprises a sensor in a form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

In some embodiments, the at least one sensor assembly comprises one or more chemically sensitive sensors and a processing unit comprising a learning and pattern recognition analyzer configured for receiving sensor output signals and comparing the signals to a stored data, e.g., by utilizing a pattern recognition algorithm.

In some embodiments, the sensor is provided in the form of a plurality of nanoparticles associated to a surface. The sensor surface may comprise one or more sensing regions, each of the regions being associated with same or different population of nanoparticles, such that a signal may be independently derived from each of the sensing areas, and be indicative of an interaction (or lack thereof) between VCs present in the sample and the nanoparticles on the sensing regions.

In some embodiments, the sensor assembly is selected to be responsive and interact with VCs characteristic of bacterial infections of the respiratory tract.

In some embodiments, the sensor assembly is selected to be responsive and interact with VCs characteristic of bacterial infections of the respiratory tract and substantially unresponsive or less or differently responsive to VCs characteristic of viral infections.

In some embodiments, the sensor assembly is selected to be responsive and interact with VCs selected from ethanol, methanol, 2-butanol, pentanol, 2-methyl-1-propanol, 3-methyl-1-butanol, 2-methyl-butanal, 1-undecane, 2,4-dimethyl-1-heptane, 2-butanone, 2-propanol, 4-methyl-quinazoline, 1-octanol, ethyl acetate, lactic acid, isovaleric acid, indole, hydrogen cyanide, methyl thiocyanide, 2-acetophenone, ammonia, methylthiocyanide, 2,2,4,4-tetramethyloxolane, methyl-4-methylpentanoate, 4-methyl pentanoic acid, 1-methyl-2-(1-methylethyl)-benzene, cymol, 4-methyldodecane, methyl nicotinate, 1,2-bis(trimethylsily)-benzene, gamma-butyrolactone, 3Z-octenyl acetate, 3-methylcyclo hexene, superoxide anion (O₂·—), hydroxyl radicals (OH·), singlet oxygen (O₂·), hydrogen peroxide (H₂O₂), hypochlorous acid (HOCI) and myeloperoxidase (MPO).

In some embodiments, the sensor is in a form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

In some embodiments, the sensor assembly is or comprises nanoparticles

Each of the sensing regions present on the sensor surface comprises a plurality of nanoparticles of a particular population, wherein each population differs from another in at least one of particle size, particle morphology (e.g., core/shell particles, non-core/shell particles, spherical, cubic, tetrahedral, triangular, dumbbell, elongated, multiparticles or fused particles, etc), particle composition (e.g., doping, metallic particles, non-metallic particles, conductive particles, novel metal particles, hybrid materials, etc), surface decoration (e.g., presence of material islands, association with ligand groups, etc) and others.

In some embodiments, the sensor surface comprises one or more sensing regions, each of the regions being associated with same or different population of nanoparticles, wherein a signal independently derived from each of the sensing areas is indicative of an interaction (or lack thereof) between VCs present in the sample and the nanoparticles on the sensing regions.

In some embodiments, each of the sensing regions comprises a plurality of nanoparticles of a particular population, wherein each population differs from another in at least one of particle size, particle morphology, particle composition, and surface decoration.

In some embodiments, each sensing region comprises a different selection of nanoparticles. In some embodiments, each sensing region comprises a mixed population (an inhomogeneous population) of nanoparticles, while in other embodiments, each sensing region comprises a uniform population (a homogenous population) of nanoparticles.

In a plurality of such sensing regions, one or more thereof may comprise a plurality of particle populations, namely an inhomogeneous population of particles, wherein some of the nanoparticles differ in structure, others in composition and still others in surface decoration. For example, a sensing region may comprise two populations of nanoparticles, one population comprising particles of one metal and another population comprises particles of a different metal. In a similar way, all particles may be of one metal but differ from each other in their surface decoration (e.g., presence of ligands or selection of ligands).

In some embodiments, the nanoparticles are core/shell particles, non-core/shell particles, spherical, cubic, tetrahedral, triangular, dumbbell, elongated or fused particles. In some embodiments, the particles are spherical in shape.

In some embodiments, the nanoparticles are metallic nanoparticles; wherein the metal is optionally selected amongst any metal of the Periodic Table of the Elements. In some embodiments, the metals are of any of Groups IIIB, IVB, VB, VIB, VIIB, VIIIB, IB and IIB of block d of the Periodic Table. In some embodiments, the metal is selected from Sc. Ti, V. Cr. Mn, Fe, Ni, Cu, Y, Zr. Nb, Tc, Ru, Mo, Rh, W, Au, Pt, Pd, Ag, Au, Al, Mn, Co, Cd, Hf, Ta, Re, Os, Ir and Hg.

In some embodiments, the metal is gold, silver, nickel, cobalt, copper, palladium, platinum or aluminum. In some embodiments, the nanoparticles are gold nanoparticles.

The metallic nanoparticles may or may not be doped or further comprise an amount of another metallic or non-metallic material. The metallic nanoparticles may be bare, namely uncoated, or coated with a plurality of surface associated ligand molecules.

Such ligand molecules may have surface anchoring groups which may vary based on, e.g., the composition of the nanoparticles. For example, where the nanoparticles are gold nanoparticles, the surface anchoring groups may be a thiol, a disulfide, an amine and others as known in the art.

In some embodiments, wherein the nanoparticles are gold nanoparticles.

In some embodiments, the pattern recognition analyzer is configured for generating a label in a form of an electronic signal indicative of a VC profile of a sample and comparing said signal to a signal representative of a VC profile of samples obtained from a non-infected subject population and an infected subject population.

In some embodiments, the device comprises a data processing unit for data communication with the sensor assembly; a data user interface unit being in data communication with the data processing unit; wherein the data processing unit comprising data relating to a control data set and is adapted to receiving from the sensor(s) information relating to presence of VCs or pattern thereof and provide an indication of presence or absence of infection.

In some embodiments, the sample is a breath sample or a headspace obtained from a blood sample, a saliva sample, a sweat sample, feces or a urine sample.

In some embodiments, the breath sample is obtained from a subject and is received through an inlet provided in the sample collecting chamber.

In some embodiments, the at least one collecting chamber is configured to separate between different aliquots of the sample.

In some embodiments, the device comprises a valve or a valve assembly configured and operable, manually, mechanically or electronically, to allow or prevent air follow into the collecting chambers or out of the chambers.

In some embodiments, the device comprises two or more sample collecting chambers, wherein one or more of the sample collecting chambers is an environment testing chamber adapted with one or more sensors providing an initial reading of environmental parameters.

In some embodiments, the one or more sensors are configured for providing a reading relating to any one of gas composition, carbon dioxide presence and concentration, humidity and sample temperature.

In some embodiments, wherein the sample collecting chamber is configured to receive a gaseous sample while disconnected from the at least one sensor assembly.

In some embodiments, wherein the sample collecting chamber is provided under vacuum.

In some embodiments, wherein the sample collecting chamber is configured to connect to a pump.

In some embodiments, wherein the closed loop channel assembly having at least one outlet operable to exhaust the sample upon demand.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 present the LDA first canonical values of a dataset in a box plot.

### DETAILED DESCRIPTION OF EMBODIMENTS

CLINICAL STUDY—Volatile Biomarkers Signatures in Exhaled Air Samples from Subjects with and without Streptococcal pharyngitis

The aim of the study was to collect and evaluate data of potential volatile biomarkers in exhaled air of subjects with and without Strep throat infection by a method and a device of the invention. Patients presenting with symptoms of strep pharyngitis were enrolled. Classification to the 2 study arms was based on Strep rapid test and Strep culture.

Breath samples were exposed to a sensor array. Sensor's resistance was recorded at baseline-before exposure, during exposure to breath sample, and during sensor's cleaning.

The dataset included sensor's signals from 41 breath samples taken from 41 subjects tested-4 breath samples from 4 Strep A positive subjects and 37 breath samples from 37 Strep A negative subjects. Several features from the sensor's signals were extracted and a Linear discriminative analysis was performed (LDA).

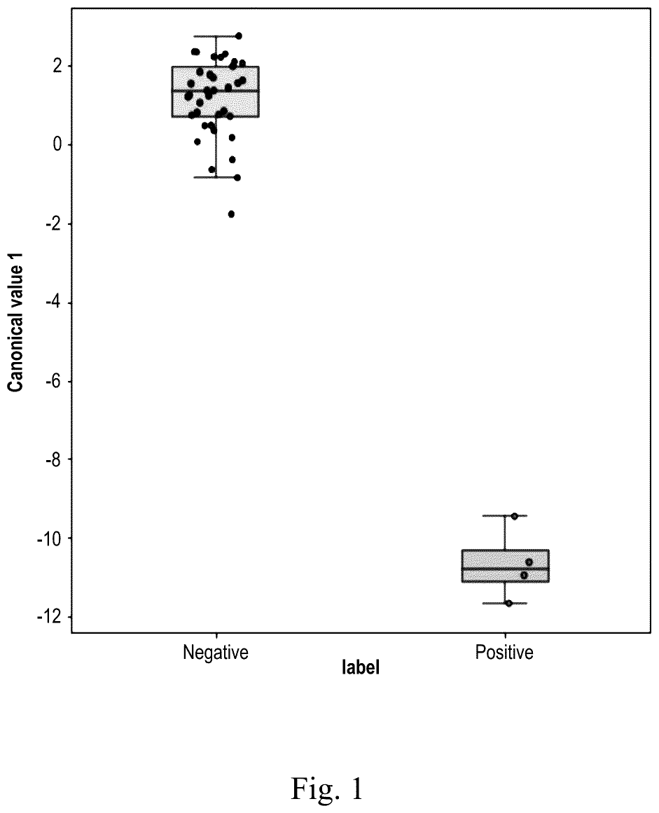

The LDA first canonical values of the dataset are presented in the box plot in FIG. 1. The horizontal line in the box represents the Median value; Each box represents Interquartile Range (IQR) for 25-75 percentiles. Black outer lines represent Min=Q1−1.5*IQR and Max=Q3+1.5*IQR. The box on the upper left side represents the 37 negative samples while the box on the lower right side represents the 4 positive samples.

For the given dataset a clear separation is visible between the Strep A negative and positive samples after LDA transformation. This supports the ability of a method and device of the invention in differentiating between subjects having an UTRI and those who do not.

1-72. (canceled)

73. A method of determining a respiratory tract bacterial infection in a subject, the method comprising:
a) exposing a gaseous sample being a breath sample or a headspace sample comprising volatile compounds (VCs) to a sensor responsive to interaction with the volatile compounds;
b) detecting/measuring an output signal received from the sensor correlating with an interaction between the VCs and the sensor; and
c) determining presence of a pattern of volatile compounds indicative of bacterial infection,
wherein the presence of a pattern of volatile compounds indicative of bacterial infection is determined using a learning and pattern recognition algorithm enabling learning, dimensionality reduction, classification, regression, optimization and pattern recognition.

74. The method according to claim 73, wherein the algorithm is selected from artificial neural network algorithms (ANN), gradient descent, spike timing dependent plasticity (STDP), principal component analysis (PCA), multi-layer perception (MLP), generalized regression neural network (GRNN), convolutional neural networks (CNN), spiking neural networks (SNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, k nearest neighbors (KNN), k means clustering (K means), spectral clustering, support vector machine (SVM), logistic regression, random forest and naïve Bayes.

75. The method according to claim 74, wherein the algorithm is LDA.

76. The method according to claim 73, wherein the method comprises obtaining a device comprising
a sample collecting chamber;
at least one sensor assembly comprising one or a plurality of sensing regions, wherein the assembly is in gaseous communication with said sample collection chamber;
a closed loop channel assembly for directing said sample from the sample collecting chamber to the at least one sensor assembly and for circulating said sample from the sample collecting chamber over the at least one sensor assembly over a period of time; and
a pattern recognition analyzer;
and carrying out the method on said device.

77. The method according to claim 73, wherein the sensor is at least one sensor assembly.

78. The method according to claim 73, wherein the sensor comprises one or more chemically sensitive sensors and a processing unit comprising a learning and pattern recognition analyzer configured for receiving sensor output signals and comparing the signals to a stored data.

79. The method according to claim 73, wherein the sensor or sensor assembly is selected to be responsive and interact with VCs characteristic of bacterial infections of the respiratory tract.

80. The method according to claim 79, wherein the sensor or sensor assembly is selected to be responsive and interact with VCs characteristic of bacterial infections of the respiratory tract and substantially unresponsive or less or differently responsive to VCs characteristic of viral infections.

81. The method according to claim 73, wherein the sensor is in a form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

82. The method according to claim 73, wherein the sensor is provided in the form of a plurality of nanoparticles associated to a surface, wherein the sensor surface comprises one or more sensing regions, each of the regions being associated with same or different population of nanoparticles, such that a signal may be independently derived from each of the sensing areas, and be indicative of an interaction (or lack thereof) between VCs present in the sample and the nanoparticles on the sensing regions.

83. The method according to claim 73, wherein the sample is a biological sample optionally selected from a blood sample, a urine sample, feces, a sweat sample and a sample of saliva, and wherein the VCs are measured in a headspace of said sample.

84. The method according to claim 73, for determining onset, presence or evolution of at least one bacterial infection of the upper respiratory tract or the lower respiratory tract, or for determining onset, presence or evolution of at least one bacterial infection induced by Streptococcal pyogenes.

85. A device being optionally a hand-held device for determining presence of a bacterial infection in a subject, the device comprising
a sample collecting chamber;
at least one sensor assembly comprising one or a plurality of sensing regions responsive to interaction with volatile compounds present in a sample obtained from the subject, wherein the assembly is in gaseous communication with said sample collection chamber;
a closed loop channel assembly configured for directing said sample from the sample collecting chamber to the at least one sensor assembly and for circulating said sample from the sample collecting chamber over the at least one sensor assembly over a period of time; and
a pattern recognition analyzer.

86. The device according to claim 85, wherein the at least one sensor assembly comprises a sensor in the form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

87. The device according to claim 85, wherein the at least one sensor assembly comprises one or more chemically sensitive sensors and a processing unit comprising a learning and pattern recognition analyzer configured for receiving sensor output signals and comparing the signals to a stored data.

88. The device according to claim 85, wherein the at least one sensor assembly is provided in the form of a plurality of nanoparticles associated to a surface.

89. The device according to claim 85, wherein the sensor is in a form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

90. The device according to claim 85, wherein the at least one collecting chamber is configured to separate between different aliquots of the sample.

91. The device according to claim 85, wherein the device comprises two or more sample collecting chambers, wherein one or more of the sample collecting chambers is an environment testing chamber adapted with one or more sensors providing an initial reading of environmental parameters.

92. The device according to claim 85, wherein the closed loop channel assembly having at least one outlet operable to exhaust the sample upon demand.
